(19) United States
(12) Patent Application Publication
(10) Pub. No.: US 2025/0017485 A1
(43) Pub. Date: Jan. 16, 2025

(54) METHOD FOR DETECTING EQUINE BONE INJURY

(71) Applicant: TELEMEDVET PTY LTD, Ascot, Western Australia (AU)

(72) Inventors: Peter John TUALLY, Ascot, Western Australia (AU); Christopher Neil COWCHER, Ascot, Western Australia (AU)

(21) Appl. No.: 18/712,971

(22) PCT Filed: Nov. 25, 2022
(86) PCT No.: PCT/AU2022/051419

§ 371 Date: May 23, 2024

(30) Foreign Application Priority Data
Nov. 25, 2021 (AU) 2021903808

(51) Int. Cl. A61B 5/055; A61B 5/00; A61B 6/50

(52) U.S. Cl. CPC A61B 5/055; A61B 5/4504; A61B 6/505; A61B 6/508; A61B 2503/40

(57) ABSTRACT

The present invention relates to a method for detecting pathological bone activity in an equine subject using a serum biomarker, and use of the serum biomarker for diagnosing and/or preventing risk of equine bone fracture and/or lameness, particularly in race horses.

### CROSS-REFERENCE TO RELATED APPLICATION

This application claims priority from Australian Provisional Patent Application No. 2021903808, the entire contents of which are incorporated herein by cross-reference.

### TECHNICAL FIELD

The present invention relates to a method for detecting pathological bone activity in an equine subject. In particular, the present invention relates to use of a serum biomarker for diagnosing and/or preventing risk of equine bone fracture and/or lameness.

### BACKGROUND

Stress fractures are a leading cause of catastrophic limb injury and a significant contributor to morbidity and mortality in the Australian racehorse industry. The prevention of fractures and lameness and the associated welfare and economic impacts represent a global priority for the racing industry. Fractures in racehorses most commonly occur as a result of pre-existing microtraumas that accumulate when bone repair capacities are overloaded with repetitive load cycles associated with athletic training. In accordance with Wolff's law, osteoclastic and osteoblastic activity responds to repetitive loads by changing bone structure, adapting its size, shape and strength to meet its loading circumstances.

The early detection of fatigue injuries and thus horses at risk of catastrophic musculoskeletal events represents a global priority for the racehorse industry due to increased public scrutiny on animal welfare. Normal bone adapts to the stresses of training through adaptive modelling leading to the laying down of new, dense bone and matrix repair via remodelling. However, often in skeletally immature individuals, combined with insufficient rest periods, the bone may not respond quickly enough to repair itself and becomes fatigued where ultimately, and suddenly, it can fail once a tipping point has been reached. While bone fatigue will often manifest as lameness, many horses show no signs of discomfort and regions that are predisposed to traumatic events often remain clinically quiescent.

Currently, there are no techniques that can accurately determine the number of load cycles a bone can withstand prior to spontaneous failure in individual cases. Although there are various metrics for measuring bone health, none of them are a reliable early predictor of fracture susceptibility. Despite significant advances in medical imaging technologies over the past decade, there exists no practical platform to serially assess the early pathognomonic features of bone disease in the horse.

In addition to medical imaging techniques, researchers have investigated the capacity of bone turnover markers (BTMs or biomarkers) to predict injured bone at risk for fracture. However, the available evidence remains heterogeneous and there is a general lack of consensus between various studies (Spitz and Newberg. Radiologic Clinics. 2002; 40 (2): 313-31). For example, there are uncertainties surrounding the use of BTMs for predicting fracture risk due to the large variability between horse breeds, age, physiologic maturity, and differences in analytical methodologies. For instance, whilst biomarkers such as glycosaminoglycans, collagen degradation markers, aggrecan synthesis markers, and markers for bone formation and degradation such as osteocalcin (OC) and C-terminal telopeptide of type I collagen have shown meaningful patterns in classifying injured horses, the dynamics may be population specific (Currie et al. J Med Imaging Radiat Sci. 2019; 50 (4): 477-87; Vial et al. Translational Cancer Research. 2018; 7 (3): 803-16; Budan et al. PLOS One. 2018; 13 (9): e0204423; Holmes et al. Vet J. 2014; 202 (3): 443-7). Moreover, growth in early career thoroughbreds may mask biomarker changes related to fracture and may only be useful in skeletally mature horses (Martin et al. Journal of Biomechanics. 1997;30 (2): 109-14).

Accordingly, there is an ongoing need for early detection methods of equine bone maladaptation to assist with preventive care and improve the welfare of race horses.

### SUMMARY

The present invention is predicated on the discovery that a serum biomarker, osteocalcin, is elevated in equine subjects with pathological bone activity, which places the subject at risk of bone fracture and/or lameness.

In one aspect, the present invention provides a method for detecting pathological bone activity in an equine subject comprising:
(a) measuring osteocalcin level in a biological sample obtained from an equine subject; and
(b) comparing the osteocalcin level in the biological sample to a reference value,
   wherein an elevated osteocalcin concentration in the biological sample relative to the reference value indicates the presence of pathological bone activity in the equine subject.

In another aspect, the present invention provides a method for predicting pathological bone fracture and/or lameness risk in an equine subject comprising:
(a) measuring osteocalcin level in a biological sample obtained from an equine subject;
(b) comparing the osteocalcin level in the biological sample to a reference value; and
   wherein an elevated osteocalcin level in the biological sample relative to the reference value is indicative of a positive prediction of bone fracture and/or lameness risk if the subject is exposed to increased biomechanical force.

In another aspect, the present invention provides a method for reducing risk of bone fracture and/or lameness in an equine subject comprising:
(a) measuring osteocalcin level in a biological sample obtained from an equine subject; and
(b) comparing the osteocalcin level in the biological sample to a reference value;
   wherein, if the osteocalcin level in the biological sample is elevated relative to the reference value, exposure of the equine subject to biomechanical force is reduced or substantially avoided so as to reduce the risk of bone fracture and/or lameness.

In yet another aspect, the present invention provides use of osteocalcin as a biomarker for detecting pathological bone activity in an equine subject.

In still another aspect, the present invention provides a kit for detecting osteocalcin level in an equine biological sample comprising:
(a) a device for obtaining a biological sample from an equine subject;
(b) a test for measuring osteocalcin level in the biological sample relative to a reference value.

### BRIEF DESCRIPTION OF THE DRAWINGS

Embodiments of the invention will now be described with reference to the following Figures, which are intended to be exemplary only, and in which:

FIG. 1 shows an example of regions of interest (ROIs) drawn over right hind fetlock with histographic representation of activity values.

FIG. 2 shows serum biomarkers measured in control and lame groups of horses. CTX—carboxyterminal telopeptides of type I collagen molecules; GAGs—proteoglycans and sulphated glycosaminoglycans; UC-II—collagen type II; BGLAP—osteocalcin; PIICP—procollagen II C terminal propeptide. Values are expressed as mean±SD. For statistical significance were considered values *p<0.05 (control vs. lame group).

FIG. 3 shows a comparison of geometric mean values obtained by scintigraphic imaging and references on (A) hind and (B) forelegs in lame group of horses. Values are expressed as mean±SD. For statistical significance were considered values *p<0.05,** p<0.01.

FIG. 4 shows the relative proportion of metabolically active injury sites in horses identified as having moderate (mPos) or significant (sPos) correlation in Example 2.

FIG. 5 is a bar graph the outcome of horses participating in the study described in Example 2, showing the percentage of horses who were provided with box rest, paddock rest, retirement, redirected to therapy or ruled out (r/o) due to significant pathology.

### DEFINITIONS

Unless defined otherwise, all technical and scientific terms used herein have the same meanings as commonly understood by one of ordinary skill in the art to which this invention belongs.

Unless otherwise specified, the indefinite articles “a”, “an” and “the” as used herein, include plural aspects. Thus, for example, reference to “an agent” includes a single agent, as well as two or more agents; reference to “the composition” or “formulation” includes a single composition or formulation, as well as two or more compositions or formulations; and so forth.

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

The term “biomarker” refers to a measurable characteristic that reflects the presence or nature (e.g., severity) of a physiological and/or pathophysiological state, including an indicator of risk of developing a particular physiological or pathophysiological state. Biomarkers may be present in a biological sample (e.g., a blood or interstitial fluid sample) obtained from a subject before the onset of a physiological or pathophysiological state, including a symptom, thereof. Thus, the presence of the biomarker in a sample obtained from the subject is likely to be indicative of an increased risk that the subject will develop the physiological or pathophysiological state or symptom thereof. Alternatively, or in addition, the biomarker may be normally expressed in an individual, but its expression may change (i.e., it is increased (elevated; upregulated; over-expressed) or decreased (downregulated; under-expressed) before the onset of a physiological or pathophysiological state, including a symptom thereof. Thus, a change in the level of expression of the biomarker is likely to be indicative of an increased risk that the subject will develop the physiological or pathophysiological state (or symptom thereof).

Reference herein to the “level” or “amount” of a biomarker means a quantitative amount (e.g., moles or number), a semi-quantitative amount, a relative amount (e.g., weight % or mole % within class or a ratio), a concentration, or the like. Thus, in reference to the amount or level of a biomarker, the terms encompasses absolute or relative amounts or concentrations of biomarkers in a sample, including ratios of levels of biomarkers, and odds ratios of levels or ratios of odds ratios. Levels or amounts may also be reflective of an individual subject or of cohorts of subjects, including levels in a population of subjects represented, for example as mean levels and standard deviations.

As used herein, the term “biomechanical force” in the context of an equine subject refers to any force acting on and/or generated within the equine body and includes most aspects of riding, training and racing.

In the context of the present invention, the terms “equine” and “horse” are used interchangeably and include all members of the equine family, including but not limited to horses and ponies. Unless otherwise specified, use of the terms equine and horse herein encompass all members of the equine family, including horses of any sex, breed, age, occupation, and the like.

As used herein, the term “lameness” in relation to a horse refers to an abnormality in its movement (e.g., gait or stance) caused by pain or reduced range of motion, caused by either a structural or a functional disorder of the musculoskeletal system of a horse.

As used herein, the term “obtained” means to come into possession. For example, For example, obtaining a biological sample, such as a blood sample, can include coming into the possession of a sample that has already been taken from a subject, as well as actively taking a sample from a subject.

The terms “pathological bone activity” and “pathological bone lesion” are used interchangeably herein and refer to abnormalities (e.g., microfracture or bone maladaptation) in bone activity caused by disease or trauma. Causes of pathological bone activity may include resorption of bone mass (osteoporosis), reduction of bone quality (osteomalacia, osteonecrosis), insufficient bone production (osteogenesis imperfecta, fibrous dysplasia), augmented bone resorption (giant cell granulomas, aneurysmal bone cyst), pathological bone remodelling, local bone destruction due to tumorous growths, and the like.

The terms “pathological bone fracture”, “bone fracture” and “fracture” are used interchangeably herein and refer to bone fractures that occurs without adequate trauma to fracture the bone and that are caused by a pre-existent pathological bone lesion.

The terms “radionuclide” and “radiopharmaceutical” are used interchangeably herein and have the same meaning.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “consisting of” means “consisting only of”, that is, including and limited to the integer or step or group of integers or steps, and excluding any other integer or step or group of integers or steps. The term “consisting essentially of” means the inclusion of the stated integer or step or group of integers or steps, but other integer or step or group of integers or steps that do not materially alter or contribute to the working of the invention may also be included.

The reference to any prior art in this specification is not, and should not be taken as, an acknowledgment or any form of suggestion that that prior art forms part of the common general knowledge.

### DETAILED DESCRIPTION

The present disclosure relates to methods for detecting pathological equine bone activity. In particular, the present inventors have identified a serum biomarker for detecting pathological bone activity in an equine subject, which may be indicative of the subject's susceptibility to bone fracture and/or lameness. More specifically, the present inventors identified that elevated levels of the serum biomarker osteocalcin, also referred to herein as OC or BGLAP, are related to pathological bone activity. Accordingly, measurement of OC levels in an equine biological sample may enable the diagnosis, prognosis and/or reduction of a horse's susceptibility to pathological bone fracture and/or lameness. Advantageously, the measurement of OC levels may provide a rapid, minimally invasive secondary measure for differentiating normal bone remodelling (non-pathological bone activity) from pathological bone activity (e.g., microfracture or bone maladaptation) in a range of horses. Thus, the methods of the present invention may be used to prevent or reduce the risk of bone fracture and/or lameness in horses, particularly race horses, where catastrophic bone fracture may necessitate retirement, and in the most severe cases, euthanasia of the animal.

Serum bone turnover markers (BTMs) are products of bone cell activity and are generally subdivided into three categories: bone resorption markers (e.g. C-terminal telopeptides of type I collagen and high free deoxypyridinoline), bone formation markers (e.g. bone alkaline phosphatase and osteocalcin), and osteoclast regulatory proteins. Changes in the serum concentration of various resorption and synthesis biomarkers of bone and cartilage in horses, which are detectable with a small blood sample, occur in response to increased biomechanical force. Typically, changes in such serum biomarker concentrations represent the normal repair processes of adaptation of equine bones to training and racing. Repeated or increased biomechanical forces may overcome the normal adaptation mechanisms of bone and joints leading to the accumulation of microdamage, which can ultimately lead to lameness or catastrophic fracture.

OC, also known as bone gamma-carboxyglutamic acid-containing protein (BGLAP), is synthesized by osteoblasts and is the most abundant non-collagenous protein of bone matrix. Serum concentrations of OC are higher in both physiological and pathophysiological conditions of increased bone turnover. However, OC, which is rapidly cleared by the kidneys, has a short half-life in blood and various factors such as age, gender, exercise and diet are reported to influence levels (Delmas et al. Bone. 1985; 6:329-341; Delmas et al. J Clin Invest. 1893; 71:1316-1321; Billinghurst, et al. Osteoarthritis Cartilage. 2003; 11:760-769). A lowering of OC levels over time has been observed in horses in training, which may relate to bone adaptation to loading forces in exercise (Jackson et al. Am J Vet Res. 2015; 76 (8): 679-87.). A longitudinal prospective clinical study reported a similar trend in uninjured horses, however, there was a significant increase in levels 4-6 months pre-injury in horses that were later diagnosed with either intra-articular fragmentation, tendon or ligamentous injury, dorsal metacarpal disease or stress fracture (Frisbie et al. Equine veterinary journal. 2010; 42 (7): 643-51).

Nuclear scintigraphy is commonly used for reliably assessing the impacts of repeated or increased biomechanical force. Typically, such impacts are detected with radionuclides (such as technetium 99m: ^{99m}Tc) and cross-sectional single-photon emission computed tomography (SPECT) scintigraphy. Radionuclide studies provide unique physiological insights into the metabolic activity of cells. When complexed to a bone seeking agent, such as diphosphonate-HDP or-MDP, radionuclides undergo chemisorption with the crystalline hydroxyapatite during the mineralisation phase of bone in proportion to osteoblastic activity. Tissue impairment is characterised by haemorrhage and calcification and the remodelling processes bind phosphate complexes with high affinity that can be detected with the inherently high contrast resolution of scintigraphy. The technique enables the exposure of pathophysiology in bone at a nascent stage, preceding structural abnormalities visible on conventional radiology and offers a feasible and affordable screening method for identifying stress fractures in equine patients, as large areas can be imaged in a reasonable timeframe.

The present inventors quantitatively analysed scintigraphic features of specific anatomical regions of a horse's appendicular skeleton in combination with secondary measures of musculoskeletal metabolism in blood. The concomitant investigation of multiple biosignals enabled the present inventors to discriminate pathological bone activity from normal physiological remodelling to identify high-risk bone lesions, by the identification of a relationship between increased physiologic bone turnover, as demonstrated on scintigrams, and a marker of extracellular bone and cartilage synthesis and resorption activity. Thus, the present inventors have now discovered that OC levels have a significant positive correlation with emerging bone and joint fatigue in horses, including horses of different sex, age, breed and occupation. Accordingly, the present invention provides a method for detecting pathological bone activity in an equine subject comprising:
(a) measuring osteocalcin level in a biological sample obtained from an equine subject; and
(b) comparing the osteocalcin concentration in the biological sample to a reference value,
   wherein an elevated osteocalcin concentration in the biological sample relative to the reference value indicates the presence of pathological bone activity in the equine subject.

Pathological bone activity may occur in any one or more bones of a horse. Typically, pathological bone activity in horses caused by increased biomechanical forces, such as during riding, training and racing, manifests in at least one anatomical region selected from the group consisting of the pastern, hoof, fetlock, carpi, tarsi, tibiae, stifles, femora, hip joints, lower axial skeleton and upper axial skeleton, which locations are the most common sites of fracture and lameness in race horses. Thus, elevated serum OC may be indicative of pathological bone activity in one or more of those anatomical regions and/or elsewhere throughout the skeletal structure of the horse.

Serum OC levels are detectable in biological samples obtained from equine subjects, preferably blood or interstitial fluid samples. A blood sample may be collected by any suitable method including, but not limited to, intravenous catheter, syringe and needle, Vacutainer® tube (Becton, Dickinson and Company), bleeding tubes, pinprick, and the like, preferably by a trained individual, such as a veterinary practitioner. In a preferred embodiment, a blood sample is collected by pinprick. Blood may be collected from any suitable site on the horse, most commonly from the jugular, cephalic, lateral thoracic and medial saphenous veins. In the case of blood collection by pinprick, samples may conveniently be obtained from the jugular vein. Methods of collecting interstitial fluid will also be known to those skilled in the art. In a preferred embodiment, interstitial fluid is collected by microneedles. Biological samples may be processed or unprocessed for analysis. For example, a blood sample may be treated (e.g., by centrifugation) to separate the blood serum (plasma) from cellular matter. Suitable techniques for separating blood plasma will be known to those skilled in the art. Blood samples may be processed and analysed for the purpose of evaluating the biomarkers almost immediately following collection (i.e., as a fresh sample), or they may be stored for subsequent analysis. If storage of a biological sample is desired or required, it is to be understood by persons skilled in the art that it should ideally be stored under conditions that preserve the integrity of the biomarker of interest within the sample (e.g., at −20° C.). In a preferred embodiment, the biological sample (e.g., blood or interstitial fluid) is obtained from the subject and analysed immediately after collection.

Suitable methods for the quantitative analysis of biomarker levels (amounts or concentrations) in an equine biological sample will be apparent to those skilled in the art. Preferably, measurement of serum biomarker (e.g., osteocalcin) levels is carried out semi-quantitatively or, more preferably, quantitatively, and may be done directly or indirectly. Direct measurement methods may include the use of high performance liquid chromatography (HPLC), near or mid infra-red spectroscopy or mass spectroscopy, suitable methods for which will be known to those skilled in the art. Indirect measurement includes measuring cellular responses, bound ligands, labels, or enzymatic reaction products. A preferred method for measurement of osteocalcin levels according to the present methods is an immunoassay, being a test that uses the binding of antibodies to antigens to identify and measure certain substances. Immunoassay refers to all immunological methods of measuring an analyte including radioimmunoassay (RIA), enzyme immunoassays (EIA) e.g., enzyme linked immunosorbant assay (ELISA), western blot and immunoprecipitation. In a preferred embodiment, osteocalcin levels are measured by ELISA. Immunoassays require biospecific capture reagents, such as antibodies, to capture the biomarkers. In the present invention, the OC polypeptide can be synthesized and used to generate antibodies by methods well known in the art.

In other preferred embodiment, the equine biological sample may be analysed by means of a biochip. A biochip generally comprises an insoluble substrate having a substantially planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art and are commercially available.

In some embodiments a biological sample may be collected using a dermal patch. The patch may be constructed by any means known in the art and preferably comprises a material into which the sample can migrate and/or accumulate. In a particularly preferred embodiment, the patch comprises one or more (preferably an array of) microneedles for collection of dermal interstitial fluid. The patch may further comprise a biosensor or biochip to enable rapid detection and/or continuous monitoring of OC levels. The material may bind the OC biomarker present in the interstitial fluid. The biomarker may be detected or measured in the patch either while in situ on the subject or after removal from the subject. That may be accomplished by incorporation of a detection system in the patch, for example the patch may contain an antibody specific or selective for the biomarker together with one or more detectable labels. The detectable label may be directly conjugated to the antibody. Alternatively, the detectable label can be used to label the antibody indirectly. Detectable labels for use in dermal patches labels may include but are not limited to an enzyme label, a fluorescent label, a chemiluminescent label or a bioluminescent label.

The methods disclosed herein seek to detect serum biomarker levels, particularly osteocalcin levels, in an equine subject. An “elevated” equine serum biomarker (e.g., osteocalcin) level refers to a statistically significant increase in equine serum level of the biomarker relative to a control population. By way of example, an increase in OC level of at least 10%, 25%, 50% 75%, 100%, 125%, 150%, 200%, 300%, or more, relative to a control population may be indicative of pathological bone activity, provided that increase is statistically significant. In certain embodiments, an OC concentration of greater than 0.85 ng/L in the biological sample indicates the presence of pathological bone activity in the equine subject. For example, an OC concentration or greater than 0.85 ng/L, or 0.90 ng/L, or 0.95 ng/L, or 1.0 ng/L, or more may indicate the presence of pathological bone activity in the equine subject. Statistical significance may be calculated based on relevant population parameters, examples of which may include the sex, age, breed, occupation, training regimen and size of the population. It will be understood by persons skilled in the art that, where a comparison is made to a reference value for a control population, the manner in which the equine serum sample is assessed for biomarker levels should be substantially identical to the manner in which the reference value is derived in order to ensure that an appropriate comparison can be made for the purposes of determining the presence of pathological bone activity and/or risk of bone fracture or lameness, as described herein. Alternatively, the reference value of a biomarker (e.g., osteocalcin) may be established in an autologous control sample obtained from the subject. That is, the sample is obtained from the same subject from which the sample to be evaluated is obtained.

Those skilled in the art will be able to determine the known levels of biomarker that are associated with the presence of pathological bone activity in horses. For example, the measured biomarker levels in a population of horses with pathological bone activity can be compared to a population of horses without pathological bone activity (i.e., a control or reference population). In some embodiments, a reference level may be determined above or below which a diagnosis or prediction is made. Evaluating the levels in further equine patients, e.g., in cohort studies, may help refine the reference levels and distinguish between different grades of severity of pathological bone activity or risk of bone fracture/lameness. The reference levels given in the examples disclosed herein may serve only as a first guideline to detect pathological bone activity or diagnose the risk of an individual equine subject of bone fracture and/or lameness. The person skilled in the art will be able to determine other reference levels. The value of the reference level may also depend on the desired sensitivity and specificity of the diagnosis. That level can be determined empirically or through the use of statistical methods.

Advantageously, the methods of the present invention may be used to determine whether an equine subject is at risk of bone fracture and/or lameness (i.e., whether the subject is likely to develop bone fracture and/or lameness), for example, if exposed to increased biomechanical force. As used herein, reference to “increased” biomechanical force encompasses maintenance of existing biomechanical forces (e.g., by maintenance of a training schedule) as well as intensified biomechanical force (e.g., by increasing training intensity or by running a horse in a race). Thus, the present invention also provides a method for predicting pathological bone fracture and/or lameness risk in an equine subject comprising:
(a) measuring osteocalcin level in a biological sample obtained from an equine subject; and
(b) comparing the osteocalcin level in the biological sample to a reference value:
   wherein an elevated osteocalcin concentration in the biological sample relative to the reference value is indicative of a positive prediction of bone fracture and/or lameness risk if the subject is exposed to increased biomechanical force.

In certain embodiments, the methods of the present invention may be particularly suitable for use in skeletally mature equine subjects. As used herein, the term “skeletally mature” when used in relation to an equine subject refers to an equine subject whose bones have reached a level of development where the configuration of the epiphyses and physeal plates have reached a certain level of ossification and closure of growth plates has occurred. For example, a skeletally mature equine subject be at least about 2 years of age (yo), e.g., at least about 2 yo, 3 yo, 4 yo or more. In an embodiment, a skeletally mature equine subject is 2 years of age or more. In an embodiment, a skeletally mature equine subject is 3 years of age or more.

It would be apparent to persons skilled in the art that the risk of bone fracture and/or lameness in an equine subject will vary, for example, from being at low (including negligible) or decreased risk of fracture and/or lameness to being at high or increased risk of fracture and/or lameness. A low or decreased risk means that the subject is less susceptible to fracture and/or lameness as compared to a subject (or population of subjects) determined to be at high or increased risk. Conversely, a subject that is at high or increased risk is a subject who is more susceptible to fracture and/or lameness as compared to a subject (or population of subjects) who is not at risk or is at low risk (including negligible risk).

Likelihood is suitably based on mathematical modelling. An increased likelihood of bone fracture or lameness, for example, may be relative or absolute and may be expressed qualitatively or quantitatively. For instance, an increased risk may be expressed as simply determining the subject's level of a given biomarker at one or more time points and placing the test subject in an “increased risk” category, based upon the corresponding reference biomarker profile as determined, for example, from previous population studies at the same time points. Alternatively, a numerical expression of the test subject's increased risk, e.g., as a percentage, may be determined based upon biomarker (i.e., osteocalcin) level analysis.

In some embodiments, likelihood is assessed by comparing the level or abundance of biomarker (i.e., osteocalcin) to one or more preselected levels, also referred to herein as threshold or reference levels. Thresholds may be selected that provide an acceptable ability to predict risk. In illustrative examples, receiver operating characteristic (ROC) curves are calculated by plotting the value of a variable versus its relative frequency in two populations in which a first population is considered at risk of fracture and/or lameness and a second population that is not considered to be at risk, or have a low risk, of fracture and/or lameness (called arbitrarily, for example, “low risk” and “high risk”).

In some embodiments, an equine subject is considered at risk of fracture and/or lameness where the osteocalcin level in a biological sample from the subject is elevated as compared to the corresponding biomarker in a healthy subject, as described herein, particularly a healthy subject of comparable characteristics, such as sex, age, breed and occupation.

For any particular biomarker, such as OC, a distribution of biomarker levels or activities for subjects with a first condition as compared to subjects with a second condition (e.g., low risk vs. high risk) will likely overlap. Under such conditions, a test does not absolutely distinguish the first condition and the second condition with 100% accuracy, and the area of overlap indicates where the test cannot distinguish between these conditions. A threshold is selected, above which (or below which, depending on how a biomarker changes between the first and second conditions) the test is considered to be “positive” and below which the test is considered to be “negative”. The area under the ROC curve is a measure of the probability that the perceived measurement will allow correct identification of a condition (see, e.g., Hanley et al., Radiology (1983) 143:29-36). Alternatively, or in addition, thresholds may be established by obtaining an earlier biomarker result from the same subject, to which later results may be compared. In these embodiments, the subject in effect acts as their own “control group”. For example, an increase in OC level over time in the same subject can indicate an increased risk of bone fracture or lameness (and vice versa).

In some embodiments, a positive likelihood ratio, negative likelihood ratio, odds ratio, or hazard ratio is used as a measure of the ability of the methods of the present invention to predict a condition, prognostic risk, or treatment outcome. In the case of a positive likelihood ratio, a value of 1 indicates that a positive result is equally likely among subjects in both a first group (e.g., high risk) and a second (control) group (e.g., low risk); a value greater than 1 indicates that a positive result is more likely in the first group; and a value less than 1 indicates that a positive result is more likely in the second group. In the case of a negative likelihood ratio, a value of 1 indicates that a negative result is equally likely among subjects in both groups; a value greater than 1 indicates that a negative result is more likely in the first group; and a value less than 1 indicates that a negative result is more likely in the second group. In the case of an odds ratio, a value of 1 indicates that a positive result is equally likely among subjects in both the first and second groups; a value greater than 1 indicates that a positive result is more likely in the first group; and a value less than 1 indicates that a positive result is more likely in the second group. In the case of a hazard ratio, a value of 1 indicates that the relative risk is equal in both the first and second groups; a value greater than 1 indicates that the risk is greater in the first group; and a value less than 1 indicates that the risk is greater in the second group.

In some cases, multiple thresholds may be determined in so-called “tertile”, “quartile”, or “quintile” analyses. In those methods, the first and second groups (or “low risk” and “high risk”) groups are considered together as a single population and are divided into 3, 4, or 5 (or more) “bins” having equal numbers of subjects. The boundary between two of these “bins” may be considered “thresholds.” The likelihood of fracture or lameness may be assigned based on which “bin” a test subject falls into.

In other embodiments, particular thresholds for the reference biomarker (i.e., osteocalcin) are not relied upon to determine if the biomarker level obtained from a subject is correlated to risk of bone fracture/lameness. For example, a temporal change in the biomarker can be used to rule in or out such risk.

As assessment of the likelihood of bone fracture/lameness in an equine subject may include an assessment of the probability of bone fracture or lameness in the subject. For example, the probability that an individual identified as being at risk of bone fracture/lameness may be expressed as a “positive predictive value” or “PPV.” Positive predictive value can be calculated as the number of true positives divided by the sum of the true positives and false positives. PPV is determined by the characteristics of the predictive methods of the present invention as well as the prevalence of the condition in the population analysed. The statistical algorithms can be selected such that the positive predictive value in a population considered to be at risk of bone fracture/lameness is in the range of 50% to 99% and can be, for example, at least 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In other examples, the probability that a subject is identified as not being at risk of bone fracture/lameness may be expressed as a “negative predictive value” or “NPV.” Negative predictive value can be calculated as the number of true negatives divided by the sum of the true negatives and false negatives. Negative predictive value is determined by the characteristics of the diagnostic or prognostic method, system, or code as well as the prevalence of risk in the population analysed. The statistical methods and models can be selected such that the negative predictive value in a population considered at risk of bone fracture/lameness is in the range of about 50% to about 99% and can be, for example, at least about 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Preferably, the prediction of risk of bone fracture and/or lameness in an equine subject according to the methods disclosed herein are expressed as a positive predictive value. For example, an elevated osteocalcin concentration in the biological sample relative to the reference value may be indicative of at least 50%, 60%, 70%, 80%, 90%, 95%, or 99% risk of bone fracture and/or lameness if the equine subject is exposed to increased biomechanical force, e.g., due to riding, training or racing. Preferably, an elevated osteocalcin concentration in the biological sample relative to the reference value is capable of indicating whether a horse is at least at 50% risk, more preferably at least at 60% risk, even more preferably at least at 70% of risk of bone fracture and/or lameness risk if the subject is exposed to increased biomechanical force. In an embodiment, the level of risk of bone fracture and/or lameness may be related to the level of elevation of OC levels in a subject relative to a reference value. For example, a 50% elevation in OC levels may correspond to a 50% increase in risk of bone fracture/lameness, a 60% elevation in OC levels may correspond to a 60% increase in risk of bone fracture/lameness, and so on. In another embodiment, the methods disclosed herein may provide a binary (e.g., low vs. high) or graded (e.g., low, medium, high, etc.) risk assessment.

Advantageously, a predictive method for bone fracture and/or lameness risk in a horse may also allow for the prevention of, or reduction in risk of, such bone fracture and/or lameness, for example, by reducing or substantially avoiding exposure of the equine subject to biomechanical force. Thus, the present invention also provides a method for reducing risk of bone fracture and/or lameness in an equine subject comprising:
(a) measuring osteocalcin level in a biological sample obtained from an equine subject;
(b) comparing the osteocalcin level in the biological sample to a reference value:
   wherein, if the osteocalcin concentration in the biological sample is elevated relative to the reference value, exposure of the equine subject to biomechanical force is reduced or substantially avoided so as to reduce the risk of bone fracture and/or lameness. Substantial avoidance of biomechanical force may, for example, include minimising exposure of the horse to biomechanical force, e.g., by resting, grazing and/or stabling the horse as an alternative to riding, training or racing.

The methods of the present invention may further comprise preventative action to reduce the risk of fracture or lameness in the horse. For example, the risk of bone fracture and/or lameness may be further mitigated by repeating the above method at one or more periods of time following the initial test, until the osteocalcin concentration in the biological sample obtained in step (a) is no longer elevated relative to the reference value, before exposing the equine subject to increased biomechanical force. For example, the method may be repeated at fixed or regular intervals, e.g., weekly, monthly, bimonthly, quarterly, yearly, or more, until such time as there is no statistically significant elevation of OC serum level relative to the reference value. Alternatively, the method may be repeated until such time as a veterinary practitioner, trainer, or other suitable person deems the risk of bone fracture and/or lameness to be at an acceptable level to expose the horse to increased biomechanical force. Acceptable levels of risk may be determined as discussed above on a case-by-case basis by a person skilled in the art, preferably a veterinary practitioner or trainer. Alternatively, pre-determined acceptable risk levels may be defined, for example, such that the serum osteocalcin level is less than 50%, or less than 40%, or less than 30%, or less than 20%, or less than 10% or less than 5%, or less than 2%, or less than 1% elevated relative to the reference value. Preferably, there is no statistically significant difference in serum osteocalcin level between a biological sample of an equine subject and a reference (control sample) before the horse is exposed to increased biomechanical force. In other instances, a pre-determined period of time, e.g., 1 month, 3 months, 6 months, 12 months, or more, may be decided by a skilled person, preferably a veterinary practitioner, after which the risk of bone fracture and/or lameness is deemed to be at an acceptable level to expose the horse to increased biomechanical forces.

The methods for reducing risk of bone fracture and/or lameness in an equine subject as disclosed herein may further include therapeutic interventions for managing the risk of bone fracture and/or lameness in an equine subject. The management of said risk can include identification and amelioration of the underlying cause and use of therapeutic agents or treatment regimens for preventing or delaying the onset of bone fracture and/or lameness. In addition to resting a horse to allow the bone to repair, treatment regimens may further include physiotherapy support, ultrasound shockwave for adjacent soft tissue, medication, or other appropriate treatments as determined by a veterinary practitioner. In some embodiments, veterinary compositions (medications) may be administered to the equine subject in an effective amount to achieve their intended purpose. The dose of any active compounds administered to the subject should be sufficient to achieve a beneficial response in the subject. Suitable veterinary compositions and treatment regimens will be apparent to those skilled in the art.

Thus, the present invention also extends to preventing or delaying the onset of bone fracture and/or lameness in an equine subject comprising:
(a) selecting an equine subject identified as being at risk of bone fracture and/or lameness according to the method of the present invention; and
(a) exposing the equine subject to a treatment regimen for preventing or delaying the onset of bone fracture and/or lameness in the equine subject.

Following diagnosis, the treatment regimen to be adopted or prescribed may depend on several factors, including the age, weight and general health of the horse. Another determinative factor may be the degree of risk of bone fracture and/or lameness determined by OC levels in accordance with the present invention. For instance, where the subject is determined to be at high risk of bone fracture and/or lameness, a more aggressive treatment regimen may be prescribed as compared to a subject who is determined to be at low risk of bone fracture and/or lameness.

The methods disclosed herein may be particularly useful for preventing bone fracture and/or lameness in race horses. For example, in some embodiments the methods disclosed herein may be readily performed trackside to identify race horses at risk of bone fracture and may assist with informing the decision whether or not to run the horse in a race (i.e., whether or not to scratch the horse). The methods disclosed herein may provide a useful measure of the level of risk of a bone fracture or lameness in the horse if included in the race. As discussed above, risk may be measured in a binary (e.g., low vs. high risk) or graded manner based on individual or population data. In a preferred embodiment, a percentage risk of fracture or lameness may be assigned to a horse using the methods disclosed herein to assist with deciding whether a horse should enter a race. For example, a horse identified as having at least a 50%, or 60%, or 70%, or more risk of suffering a bone fracture or lameness using the methods disclosed herein may be scratched from a race so as to prevent catastrophic fracture or lameness that might otherwise require the horse to be retired or, in severe cases, euthanized.

the present invention also provides kits for detecting osteocalcin level in an equine biological sample comprising:
(a) a device for obtaining a biological sample from an equine subject;
(b) a test for measuring osteocalcin level in the biological sample relative to a reference value.

The device for obtaining a biological sample may be any suitable device, including a syringe and needle, catheter or, preferably, a pinprick device or microneedles (e.g., as part of a subcutaneous flash interstitial fluid monitor chip). Similarly, the test for measuring osteocalcin level in the biological sample may be any suitable test, including a test described elsewhere herein, such as ELISA or mass spectrometry. For example, the kit may include an element that is essential for performing an ELISA test. The ELISA kit may include an antibody that is specific for osteocalcin and an agent that can be used to measure the level of the biomarker. The ELISA kit may include a reagent that can detect an attached antibody, such as a labeled secondary antibody, chromophores, an enzyme (e.g., an enzyme conjugated to antibody), and other substances that can bind to the substrate or antibody thereof. Further, it may include an antibody that is specific for a quantitative control group.

Preferably, the kits disclosed herein further comprise a data processing and output device, wherein such device is capable of comparing the OC concentration in the biological sample to a reference sample, and providing an output from which the presence of pathological bone activity can be detected. In some embodiments, the kits disclosed herein may comprise a physical reference sample for comparison with the biological sample or, more preferably, predetermined reference values are programmed into the data processing and output device for automated comparison. The device for obtaining a biological sample from an equine subject, the test for measuring OC level in the biological sample and, optionally, the data processing/output device may be contained within the same or different devices.

The kits disclosed herein are ideally suitable for predicting risk of fracture or lameness. Such risk may be assessed by manual operator extrapolation from the output data or, preferably, by providing an automated risk assessment. Such assessment may, for example, be carried out using a pre-programmed algorithm or by artificial intelligence. Preferably, the kits are suitable for trackside use for detecting OC levels in a race horse. For example, the kits are preferably compact, portable and provide rapid data output.

In one embodiment, the kit comprises an insoluble support, such as a chip, a microtiter plate or a bead or resin having a capture reagent attached thereto, wherein the capture reagent binds osteocalcin. Thus, for example, the kits of the present invention may comprise mass spectrometry supports, such as ProteinChip R arrays. In the case of biospecfic capture reagents, the kit may comprise an insoluble support with a reactive surface and a container comprising the biospecific capture reagent such as an antibody, aptamer, affibody, diabody, minibody or fragments thereof.

In an embodiment the kit comprises a washing solution or instructions for making a washing solution, in which the combination of the capture reagent and the washing solution allows capture of the biomarkers or biomarkers on the solid support for subsequent detection by, e.g., mass spectrometry or ELISA. The kit may include more than one type of capture reagent, each may be present on a different solid support. In another embodiment, the kit comprises one or more containers with biomarker samples, to be used as standard for calibration.

The kit can also feature printed instructions for using the kit to qualitatively or quantitatively determine one or more biomarkers of the present invention.

The methods of the present invention, as broadly described herein, may permit the generation of high-density data sets that can be evaluated using informatics approaches. High data density informatics analytical methods are known and software is available to those in the art, e.g., cluster analysis (Pirouette, Informetrix), class prediction (SIMCA-P, Umetrics), principal components analysis of a computationally modelled dataset (SIMCA-P, Umetrics), 2D cluster analysis (GeneLinker Platinum, Improved Outcomes Software), and metabolic pathway analysis (biotech.icmb.utexas.edu). The choice of software packages offers specific tools for questions of interest (Kennedy et al., Solving Data Mining Problems Through Pattern Recognition. Indianapolis: Prentice Hall PTR, 1997; Golub et al., (2999) Science 286:531-7; Eriksson et al., Multi and Megavariate Analysis Principles and Applications: Umetrics, Umea, 2001). In general, any suitable mathematic analyses can be used to evaluate one or more biomarkers in a biomarker profile with respect to determining the likelihood that the subject is at risk of bone fracture or lameness. For example, methods such as multivariate analysis of variance, multivariate regression, and/or multiple regression can be used to determine relationships between dependent variables (e.g., clinical measures) and independent variables (e.g., levels of biomarkers). Clustering, including both hierarchical and non-hierarchical methods, as well as nonmetric Dimensional Scaling can be used to determine associations or relationships among variables and among changes in those variables.

In some embodiments, a biomarker profile is used to assign a risk score which describes a mathematical equation for evaluation or prediction of risk. The evaluation of risk may also take into account genotype and other clinical or phenotypic features, such as age.

In addition, principal component analysis is a common way of reducing the dimension of studies, and can be used to interpret the variance-covariance structure of a data set. Principal components may be used in such applications as multiple regression and cluster analysis. Factor analysis may be used to describe the covariance by constructing “hidden” variables from the observed variables. Factor analysis may be considered an extension of principal component analysis, where principal component analysis is used as parameter estimation along with the maximum likelihood method. Furthermore, simple hypothesis such as equality of two vectors of means can be tested using Hotelling's T squared statistic.

In some embodiments, the data sets corresponding to biomarker profiles are used to create a diagnostic or predictive rule or model based on the application of a statistical and machine learning algorithm. Such an algorithm uses relationships between a biomarker profile and risk of bone fracture and/or lameness observed in control subjects or typically cohorts of control subjects (sometimes referred to as training data), which provides combined control or reference biomarker profiles for comparison with biomarker profiles of a subject. The data are used to infer relationships that are then used to predict the status of a subject and the presence or absence of risk of bone fracture and/or lameness.

Persons skilled in the art of data analysis will recognize that many different forms of inferring relationships in the training data may be used without materially changing the present invention.

The present inventors have found that measurement of OC levels in an equine biological sample may be suitable for detecting pathological bone activity, thereby providing a relatively simple, low cost and minimally invasive test for predicting and/or preventing risk of bone fracture or lameness in horses, particularly race. In contrast to previous biomarker studies, the methods of the present invention may be suitable for use in a variety of horses, for example, horses of difference sexes, ages, breeds (e.g., thoroughbred, standardbred, warmblood, quarter horse), occupations (e.g., racing, trotting, eventing, pleasure), training regimens and the like. Further, use of the methods of the present invention as a diagnostic tool may, for example, avoid the need for exposure of horses and their handlers to the high levels or radiation associated with nuclear scintigraphy as a first line of diagnosis. Alternatively, the methods disclosed herein may also be carried out in conjunction with (i.e., before or after) at least one medical imaging technique, e.g., a radiographic, scintigraphic or magnetic resonance imaging (MRI) technique, preferably SPECT or SPECT/CT imaging. For example, the methods disclosed herein may be performed prior to conducting medical imaging techniques to determine whether such techniques are indicated in a particular equine subject. Additionally or alternatively, such imaging techniques may desirably be used in conjunction with the methods for detecting pathological bone activity disclosed herein, for example, as a confirmatory method and/or to determine the nature and/or location of the pathological bone activity. The use of additional medical imaging techniques in conjunction with the methods of the present invention may also assist with increasing the predictive power of the risk of bone fracture or lameness of an equine subject.

Those skilled in the art will be aware that the invention described herein is subject to variations and modifications other than those specifically described. It is to be understood that the invention described herein includes all such variations and modifications. The invention also includes all such steps, features, methods, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Example 1. Identification of Serum Biomarker

### Horse Samples

Twelve horses were referred for scintigraphic investigation of lameness (Table 1), of which nine were specifically referred for investigation of hindlimb lameness and underwent scanning of the rear legs, lower lumbar spine, pelvis and sacrum, combined with 3D SPECT imaging. The scan series for the remaining three horses was limited to the front legs, shoulder region and 2D planar imaging of the cervico-thoracic spine. Blood samples from three sets of clinically sound thoroughbred horses that were matched by age, breed and occupation were also included and these represented controls. Three sets of 7-year-old standardbred samples and single samples of the younger standardbreds were also included. All animal procedures related to this study were approved by the University of Western Australia Animal Ethics Committee and Biosafety Office (UWA RA/3).

TABLE 1
Age, breed and occupation of horses referred for scintigraphy
Lame horses for investigation

| | Age range | | | |
|---|---|---|---|---|
| Breed | (years) | Occupation | Number | Sex |
| Thoroughbred | 2-4 | Racing | 7 | 3 = Gelding |
| Standardbred | 2-7 | Trotting | 3 | 2 = Gelding |
| Warmblood | 7 | Eventing | 1 | Gelding |
| Quarter Horse | 5 | Pleasure | 1 | Mare |

### Data Analysis

All presented data are shown as mean±standard deviation (SD). Figures and tables were made in Prisma 8 for macOS version 8.4.0. Comparison between serum biomarkers in lame and control groups was determent after normality test by Wilcoxon and Paired t test. On the other hand, comparison between geometric means obtained by SPECT and references in both hind and forelegs in lame horses was determined after normality test by Two-way ANOVA test. P values less <0.05 were considered statistically significant, while <0.01were considered as highly statistically significant. Correlation tests were done between parameters of interest via Pearson test where r>0 was considered as positive correlation.

### Blood Sampling

Following admission and the identification of the horse, a jugular catheter was inserted by veterinary staff and a blood sample collected into 4 ml serum collection tubes and transported to the University of Western Australia within 1 hour of collection. Samples were then centrifuged at 4000 rpm for 5 mins and serum was separated into 1 ml aliquots into 1.5 ml Eppendorf tubes (3-4 aliquots/horse) and stored at −20° C.

### Determination of Serum Biomarkers

Commercially available enzyme linked immunosorbent assay (ELISA) kits (MyBioSource, Inc. San Diego, CA) were used to ascertain the serum concentrations of the following biomarkers:
1. Carboxy-terminal telopeptides of type I collagen molecules (CTX)
2. Protoglycans and sulfated glycosaminoglycans (GAGs)
3. Collagen Type II (UC-II)
4. Osteocalcin (OC or BGLAP)
5. Procollagen II C-terminal Propeptide (PIICP)

All procedures were performed according to manufacturer guidelines.

### Imaging

Nuclear scintigraphy was conducted by scientists and veterinarians licenced to perform equine studies in accordance with the Radiation Safety Act, Western Australia, at registered premises for the handling and storage of unsealed and sealed radioactive sources. The scanners (GE healthcare, Millennium MPR SPECT system), fitted with low energy high resolution collimators designed for bone and joint imaging underwent rigorous quality control procedures prior to acquisition in accordance with manufacturer specification to ensure that optimal photopeak and uniform detection statistics across the scanner face was achieved.

Shortly after the collection of serum biomarkers, 1GBq/100 kg body weight of ^{99m}Tc-hydroxymethylene diphosphonate (HDP) was infused intravenously followed by 20 mls of normal saline and the horse remained in a designated radiation isolation stall for a sufficient time to allow for radiotracer concentration into bone (2-3 hours). Approximately 30 minutes before scanning commenced the horse received 200 mg of furosemide intravenously via the indwelling jugular catheter to facilitate faster renal excretion of the radionuclide and better target to non-target ratio of images. The horse was then led into an adjacent scanning room and sedated with 5 mg of butorphanol tartrate and 5 mg of detomidine hydrochloride i.v. and positioned for scan acquisitions with 2D phase planar imaging of either the fore or hind limbs followed by 3D SPECT of the lumbosacral spine). Based on clinical assessment of the likely source of lameness, multiplanar views of the pastern and hoof, fetlock, carpi, tarsi, tibiae, stifles, femora, hip joints and lower or upper axial skeleton were obtained. Acquisition parameters were 0.2 sec/frame dynamics for 90 seconds with a 128 matrix. Careful attention was made to ensure a constant distance from the camera face to the horse for each image was maintained.

After the scan was completed, each horse was kept in a bespoke radiation isolation stall, for 24 hours during which time radiation safety guidelines were strictly enforced/adhered to. Following the 24 hour isolation period in the lead shielded stall, the horse was discharged to the owner following a clinical assessment by veterinarian staff. The management of biological waste was conducted in accordance with the centre's Radiation Management Plan, as approved the Radiological Council of Western Australia.

### Image Analysis

The raw imaging data was transferred to GE Healthcare Xeleris V3.1 and V4processing systems in Digital Imaging and Communications in Medicine (DICOM®) standard format for both automatic and manual motion correction. The composite was generated from the corrected data for final visual interpretation of scans by experienced scientists and radiologists via the teleradiology software (SepStream™).

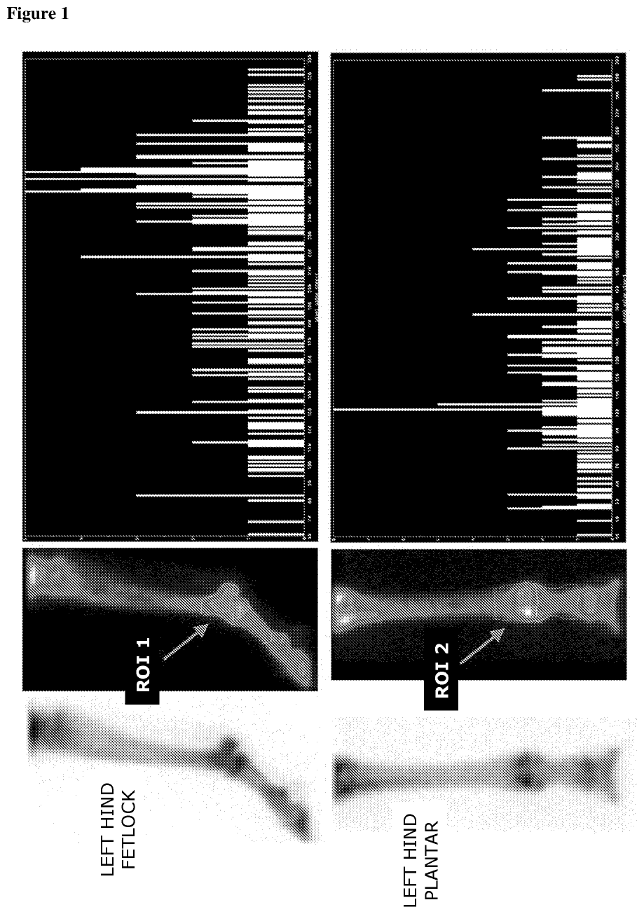

Quantitative analysis of a decay corrected, geometric mean of the average per pixel radionuclide uptake value was undertaken after manually applying discrete regions of interests (ROI) for various cortical and trabecular sites of long bones, epiphyseal, diaphyseal and metaphyseal segments, whole joints and soft-tissue regions (FIG. 1). For each horse, separate ROIs were manually drawn by three independent nuclear medicine technologists with the assistance of two experienced equine veterinary specialists. This was performed on the same type of Xeleris processing system and the mean values were used for processing (image reconstructions and data analysis).

The geometric mean is routinely adopted in nuclear medicine practice to perform planar imaging quantification and calculate the internal radiotracer uptake values of a region. It is a conjugate view method which represents the square root of the product of two quantities collected from ROIs that have been placed over an object, after background activity has been subtracted. The geometric mean corrects for attenuation of gamma rays traversing underlying tissue from opposing views and positional variation with different projections of the same anatomical area. This quantitative data is useful in augmenting qualitative interpretation of patterns of tracer uptake in organs and to accurately measure alterations in radiotracer kinetics.

$$Geometric mean = \sqrt{( {ROI}_{1} - {BKG}_{1} ) \cdot ( {ROI}_{2} - {BKG}_{2} )}$$

### Where:

ROI₁=activity corrected measured counts from a region placed over image (e.g., plantar fetlock)

ROI₂=activity corrected measured counts from a region placed on the same anatomical region in a different projection (e.g., lateral fetlock)

BKG=background activity derived soft tissue regions adjacent to the anatomical site of interest.

Radioactive values were estimated by converting collected counts with a standard correction factor based on a known source of activity concentration (MBq/mL), acquired at a set distance from the scanner face prior to the commencement of scanning. Whist use of this standard will not generate an absolute quantitative measurement, the method was considered suitable for the purposes of this pilot project and is an established method for producing clinical correction factors in human scintigraphy.

Semi-quantitative relative uptake ratios were then calculated by dividing the geometric mean values for each anatomical region with the mean values of a ROI placed over the midshaft of femora or humeri depending on whether the fore or hindlimbs were acquired. These regions were reported as normal and considered suitable for used a reference given the very low incidence of bone fatigue injury at these sites. Contralateral limb indices for each anatomical site are presented in Table 2.

TABLE 2
Activity corrected ROI indices relative to mid humeral or femora references
SCINTIGRAPHIC RELATIVE UPTAKE RATIOS BY SITE (Left/Right)

| | Distal | | | Fetlock | Fetlock | | | | |
|---|---|---|---|---|---|---|---|---|---|
| Breed/Age | Tibiae | Metatarsus | Metacarpus | (Hind) | (Fore) | Tarsus | Carpus | Pastern | Hoof |
| Case 1 (SB3G) | 1.70/1.67 | 2.27/2.10 | | | 2.00/2.10 | 1.80/1.70 | | 1.75/1.71 | 1.39/1.44 |
| Case 2 (SB2M) | 2.12/2.12 | 2.08/2.09 | | 1.86/2.12 | | 2.38/2.26 | | 1.56/1.63 | 1.26/1.29 |
| Case 3 (TB3M) | 3.44/3.46 | 3.42/3.51 | | 3.84/3.54 | | 3.18/3.31 | | 2.94/2.65 | 2.08/1.90 |
| Case 4 (SB7G) | | | 0.98/2.27 ** | | 1.55/1.70 | | 1.15/1.35 | 1.45/1.45 | 2.01/1.96 |
| Case 5 (WB5G) | 2.37/1.89 | 4.12/4.24 | | 3.38/3.64 | | 2.55/2.01 | | 2.38/3.19 | 2.24/2.37 |
| Case 6 (TB4G) | 2.22/2.02 | 2.89/2.41 | | 2.62/2.31 | | 2.32/2.21 | | 2.38/1.91 | 1.57/1.28 |
| Case 7 (TB3G) | 3.62/3.99 | 2.62/4.43 * | | 2.98/4.74 * | | 3.32/4.50 \hat{ } | | 2.90/3.97 \hat{ } | 2.30/3.07 \hat{ } |
| Case 8 (TB3M) | 3.41/2.95 | 2.56/2.61 | | 2.32/2.51 | | 3.41/2.95 | | 2.56/2.24 | 2.43/2.16 |
| Case 9 (TB3G) | 3.36/2.67 | 3.41/2.78 | | 1.98/1.45 \hat{ } | | 2.76/1.95 \hat{ } | | 2.54/1.59 * | 1.68/1.52 |
| Case 10 (TB2M) | 2.63/3.44 | 3.56/3.41 | | 2.92/2.79 | | 2.86/3.38 | | 3.33/2.79 | 2.15/1.86 |
| Case 11 (QTR5M) | | | 2.15/1.86 | | 2.56/2.04 | | 2.31/2.05 | 1.87/1.54 | 1.40/1.33 |
| Case 12 (TB3G) | | | 3.54/3.22 | | 3.22/2.86 | | 2.90/2.83 | 2.60/2.70 | 2.36/2.39 |

\hat{ } Ratio Delta 30-50%,
* Ratio Delta 51-100%,
** Ratio Delta >100%

Of the 12 horses enrolled for this study, Cases 3, 7 and 9 demonstrated scintigraphic abnormalities which were reported as suspicious for contributing to lameness. A positive correlation was observed between increased concentrations of OC and the abnormal radiomic indices of those cases, whereas no significant relationship existed with the other BTM's.

Qualitative assessment of scans on all cases was performed by an experienced equine specialist, blinded to the radiomic scores. Case 4 demonstrated asymmetrical, focal marked increase in radiopharmaceutical uptake (IRU) involving the right 3 metacarpal bone, interpreted as likely bone stress injury which was consistent with the clinical presentation of foreleg lameness. Quantitatively, the differential indices in this case confirmed the asymmetrical radionuclide activity at this anatomical region indicating a 132 percent greater radionuclide uptake and a corresponding OC value of 3.54 ng/ml, which was a 4-fold increase in concentrations compared to the matched controls (Table 3). Ten months after detection of elevated OC levels, Case 4 suffered a proximal MC fracture during a race and will be retired.

Case 9 demonstrated less prominent asymmetrical IRU involving the 2 phalanx of the left hindlimb and differential indices showing a 60% increase in uptake and OC value of 1.75 ng/ml, a 2.7-fold increase. The current status of Cases 7 and 9 is unknown.

TABLE 3
Serum Bone and Cartilage Biomarkers Control Horses
MATCHED CONTROLS SERUM BIOMAKERS (range/mean)

| Breed/Age | CTX (ng/ml) | S-GAG (ng/ml) | COL2 (ng/ml) | BGLAP (ng/ml) | PIICP (ng/ml) |
|---|---|---|---|---|---|
| Thoroughbred M (2 yo) | 0.89-1.31/1.10 | 4.66-5.29/4.97 | 1.41-1.72/1.57 | 0.79-0.83/0.81 | 29.2-37.8/33.5 |
| Thoroughbred G (3 yo) | 0.26-1.86/0.95 | 2.59-6.59/4.05 | 1.01-2.76/1.70 | 0.55-1.16/0.77 | 16.0-35.5/26.65 |
| Thoroughbred M (3 yo) | 0.82-1.38/1.15 | 3.81-4.66/4.29 | 1.23/3.16/2.38 | 0.85-2.20/1.33 | 35.9-36.8/29.5 |
| Thoroughbred G (4 yo) | 0.78-1.36/1.07 | 2.83-3.08/2.95 | 0.63-1.31/0.97 | 0.44-0.48/0.46 | 17.6-22.9/20.2 |
| Standardbred M (2 yo) | \hat{ } 1,49 | \hat{ } 4.66 | \hat{ } 2.06 | \hat{ } 0.54 | \hat{ }50.09 |
| Standardbrød G (3 yo) | \hat{ } 1.15 | \hat{ } 4.66 | \hat{ } 2.18 | \hat{ } 1.10 | \hat{ }50.39 |
| Standardbred G (7 yo) | 1.04-1.30/1.17 | 4.76-6.36/5.15 | 1.91-2.65/2.21 | 0.62-1.05/0.89 | 15.0-42.84/31.71 |

G = Gelding,
M = Mare
*Quarter Horse and Warmblood controls not obtained.
\hat{ } Only single controls obtained for this type

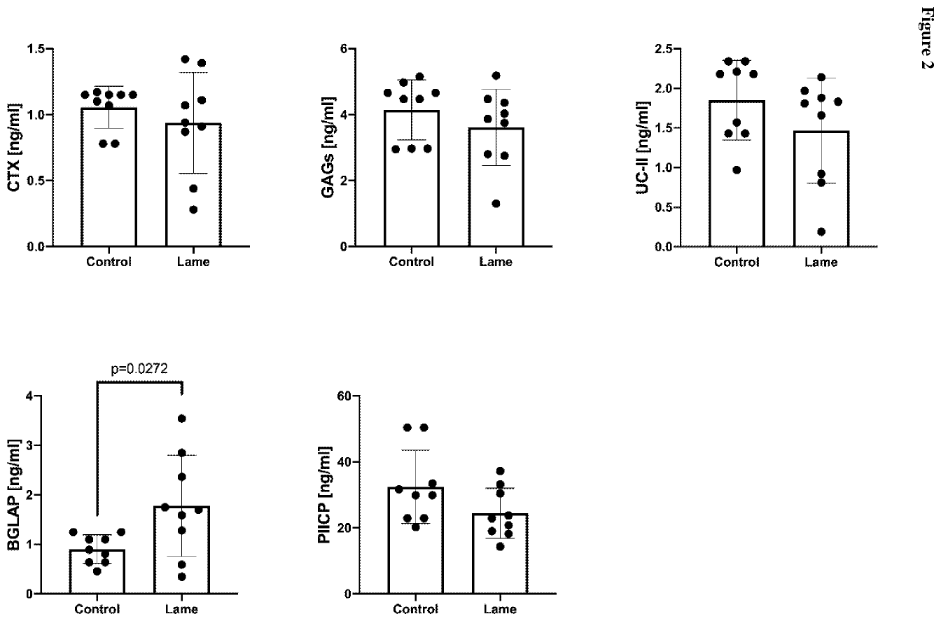

The concentration of osteocalcin (BGLAP) in the positive scans was statistically significantly increased compared to the control group, while there were no changes in other monitored serum biomarkers (Table 4; FIG. 2).

TABLE 4
Serum Bone and Cartilage Biomarkers Lame Horses.
LAME GROUP SERUM BIOMAKERS (result/control)

| Breed/Age | CTX (ng/ml) | S-GAG {ng/ml) | COL2 (ng/ml) | BGLAP (ng/ml) | PIICP (ng/ml) |
|---|---|---|---|---|---|
| Case 1 (SB3G) | 0.86/1.15 | 4.03/4.66 | 2.14/2.18 | 0.35/1.10 | 22.91/50.39 |
| Case 2 (SB2M) | 1.39/1.49 | 4.47/4.66 | 1.81/2.06 | 2.85/0.54 | 33.18/50.09 |
| Case 3 (TB3M) | 0.91/0.95 | 2.75/4,47 | 0.81/2.34 | 2.36/1.25 | 14.34/29.91 |
| Case 4 (SB7G) | 1.41/1.17 | 4.36/5.15 | 1.97/2.21 | 3.54/0.89 | 23.78/31.71 |
| Case 5 (WB5G) | 1.04/* | 4.02/* | 1.57/* | 1.74/* | 65.26/* |
| Case 6 {TB4G) | 0.28/1.07 | 1.30/2.95 | 0.19/0.97 | 0.59/0.46 | 37.25/20.23 |
| Case 7 (TB3G) | 0.44/0.78 | 2.80/2.97 | 0.92/1.43 | 1.28/0.64 | 30.48/22.91 |
| Case 8 (TB3M) | 0.94/1.15 | 5.18/4.47 | 1.66/2.34 | 1.59/1.25 | 20.81/29.5 |
| Case 9 (TB3G) | 1.11/0.78 | 3.75/2.97 | 1.83/1.43 | 1.75/0.64 | 19.05/26.65 |
| Case 10 (TB2M) | 1.07/1.10 | 3.87/4.97 | 1.88/1.57 | 1.70/0.81 | 18.19/33.48 |
| Case 11 (QTR5M) | 1.05/* | 3.77/* | 1.87/* | 1.60/* | 18.80/* |
| Case 12 (TB3G) | 0.94/0.78 | 3.66/2.97 | 1.73/1.43 | 1.48/0.64 | 18.17/26.65 |

G = Gelding,
M = Mare,
TB = Thoroughbred,
SB = Standardbred,
WB—Warmblood,
QTR = Quarter Horse
*Quarter Horse and Warmblood controls not obtained

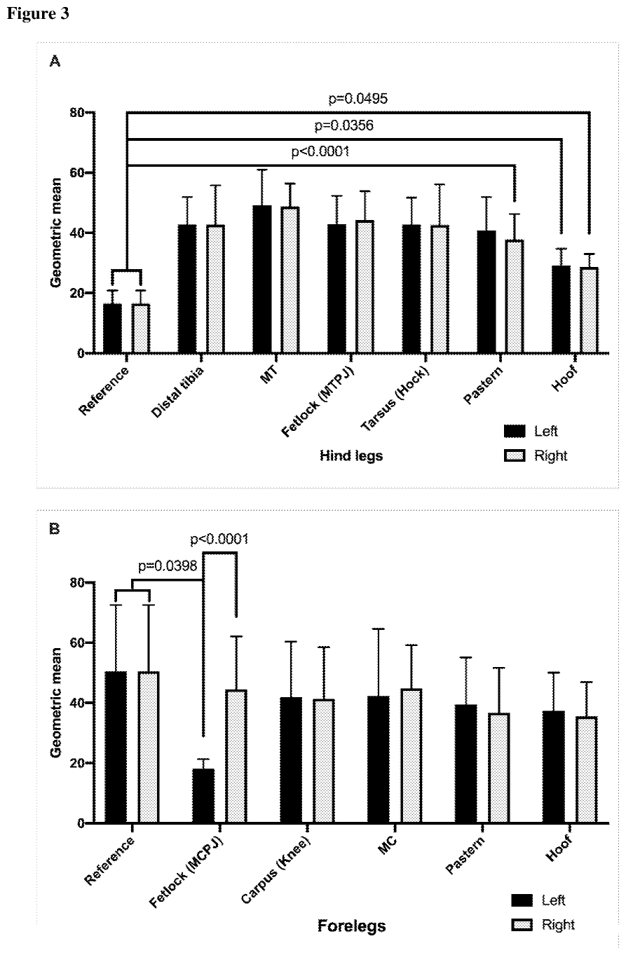

The values of the geometric mean were significantly increased in the monitored areas of the hind legs in lame horses compared to the reference values regardless of the imaged leg (FIG. 3A). Conversely, the values of the geometric mean were significantly decreased in fetlock of the left foreleg compared to the reference values and compared to the values of the right foreleg (FIG. 3B).

Strong correlations between BGLAP and ratio maintained from geometric means and reference values for L-carpus, R-carpus and L-MC were highly statistically significant (FIG. 3). Other serum biomarkers did not show correlation with any of the SPECT imagining parameters of hind and forelegs.

The results of the study described above showed that OC (osteocalcin), a marker of increased bone turnover, was statistically significantly increased in the lame group of horses. Those results are in contrast to the finding of Turlo et al. (BMC veterinary research. 2019; 15 (1): 66), where osteocalcin levels were significantly lower in injured 2 yo horses in training. OC was strongly related to the differential radiomic indices of Cases 4, 7 and 9.

Thus, the study described above provided evidence that radiomic features could be extracted to predict the risk of musculoskeletal injury/bone fracture in horses. Further, the coupling or “snapshot” of two biological signals of underlying cellular activity at the same time point helped to eliminate the possible confounders related to fluctuating concentrations of BTMs due to urinary excretion.

### Example 2. Retrospective Case Study

Blood samples were obtained and analysed in accordance with Example 1 from a cohort of 77 horses (26% Standardbreds: 74% throughbreds) and compared to the controls in Example 1. Horses were 2 yo (32%), 3 yo (46%), 4 yo (25%), 5 yo (14%), 6 yo (14%) and 7 yo (7%).

At the time of filing this application, preliminary qualitative results were obtained for 27 of the 77 horses (Table 5). The qualitative results set out in Table 5 provides a binary classification of radionuclide uptake (i.e., ⁹⁹mTc), where “0” represents a normal (Neg) correlation between radionuclide uptake and BTM concentration and “1” represents a moderately positive (mPos) or significantly positive (sPos) correlation between radionuclide uptake and BTM concentration, using a preliminary cut-off of 1 ng/L for both osteocalcin and CTX as “significant”:

### Normal (Neg)

Radionuclide uptake on the scan fits within normal limits based on the subjective assessment of the radiologist or surgeon with experience in reading nuclear medicine. That is, no cause of the lameness demonstrated on the scan.

Moderately Positive (mPos)

Radionuclide uptake on the scan is abnormal (mild or moderately higher than normal) in sites suspicious for the origin of lameness but recommends further confirmation by vets (e.g., by x-rays or blocking the area with analgesics and running the horse around to see if lameness still presents).

Strongly Positive (sPos)

Unequivocally abnormal radionuclide uptake consistent with stress fracture (markedly intense, or moderate but at a region common for fracture in horses).

TABLE 4
Binary analysis of correlation between ^{99m}Tc uptake and BTM concentration

| | | | | OC <=1 | OC 1-4 | OC >4 | CTX <1 | CTX 1-4 | CTX >4 |
|---|---|---|---|---|---|---|---|---|---|
| | Neg | mPos | sPos | ng/l | ng/l | ng/l | ng/l | ng/l | ng/l |
| 5GT | 1 | 0 | 0 | 1 | 0 | 0 | 0 | 1 | 0 |
| 5MT | 0 | 1 | 0 | 0 | 1 | 0 | 1 | 0 | 0 |
| 12GT | 0 | 1 | 0 | 0 | 1 | 0 | 1 | 0 | 0 |
| 4GT | 1 | 0 | 0 | 1 | 0 | 0 | 1 | 0 | 0 |
| 2CT | 0 | 0 | 1 | 0 | 1 | 0 | 1 | 0 | 0 |
| 3GT | 1 | 0 | 0 | 1 | 0 | 0 | 1 | 0 | 0 |
| 3MT | 0 | 1 | 0 | 0 | 0 | 1 | 1 | 0 | 0 |
| 5GT | 0 | 0 | 1 | 1 | 0 | 0 | 1 | 0 | 0 |
| 4MT | 0 | 0 | 1 | 0 | 1 | 0 | 1 | 0 | 0 |
| 8GT | 0 | 1 | 0 | 0 | 1 | 0 | 1 | 0 | 0 |
| 8GWB | 0 | 0 | 1 | 0 | 1 | 0 | 1 | 0 | 0 |
| 5GTB | 0 | 1 | 0 | 0 | 1 | 0 | 1 | 0 | 0 |
| 5GTB | 0 | 1 | 0 | 0 | 1 | 0 | 1 | 0 | 0 |
| 2CT | 1 | 0 | 0 | 0 | 1 | 0 | 1 | 0 | 0 |
| 2FT | 0 | 1 | 0 | 1 | 0 | 0 | 1 | 0 | 0 |
| 3FT | 0 | 1 | 0 | 1 | 0 | 0 | 1 | 0 | 0 |
| 2STB | 0 | 0 | 1 | 1 | 0 | 0 | 1 | 0 | 0 |
| 4GT | 0 | 1 | 0 | 0 | 0 | 1 | 1 | 0 | 0 |
| 3FT | 0 | 1 | 0 | 0 | 1 | 0 | 1 | 0 | 0 |
| 8MWB | 0 | 0 | 1 | 0 | 0 | 1 | 1 | 0 | 0 |
| 13GT | 0 | 0 | 1 | 0 | 0 | 1 | 1 | 0 | 0 |
| 12GWB | 0 | 1 | 0 | 0 | 1 | 0 | 1 | 0 | 0 |
| 7MWB | 0 | 1 | 0 | 0 | 1 | 0 | 1 | 0 | 0 |
| 6GWB | 1 | 0 | 0 | 1 | 0 | 0 | 1 | 0 | 0 |
| 9GWB | 0 | 1 | 0 | 0 | 1 | 0 | 1 | 0 | 0 |
| 3GSB | 0 | 1 | 0 | 1 | 0 | 0 | 1 | 0 | 0 |
| 5GT | 1 | 0 | 0 | 0 | 1 | 0 | 1 | 0 | 0 |

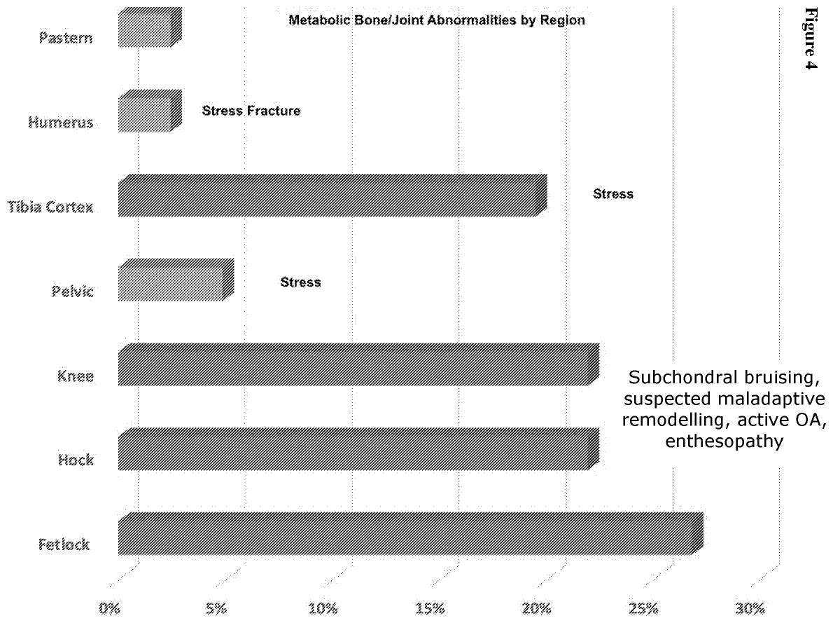

The preliminary results obtained to date indicate a strong correlation between increased osteocalcin concentration and bone/joint injuries. In particular, in 74% of the 27 cases for which qualitative results have been obtained to date, radionucleotide uptake correlated with increased OC concentrations. In the cases where a moderate (mPos) or significant (sPos) correlation was not observed based on the preliminary qualitative date, the horses were predominantly young horses (i.e., horses less than 3 yo). FIG. 4 shows the relative proportion of metabolically active injury sites in horses identified as having an mPos or sPos correlation.

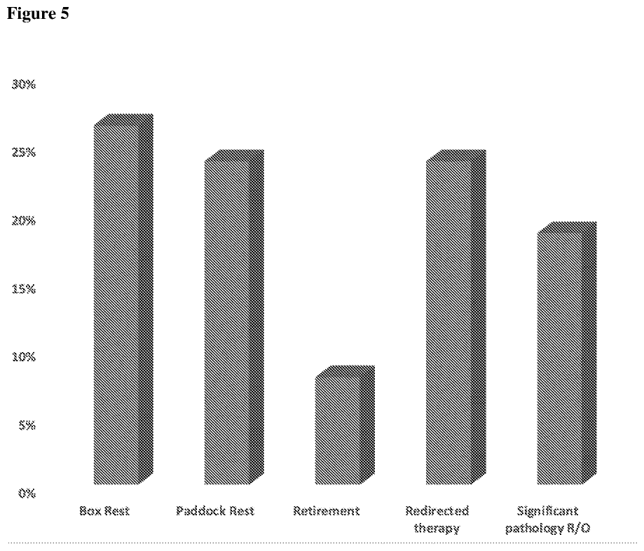

FIG. 5 shows the outcome of horses in the study (i.e., box rest, paddock rest, retirement, redirected to therapy or ruled out (r/o) due to significant pathology).

### Example 3. Prospective Case Study

Two groups of study subjects will be recruited to further investigate the correlation between serum concentrations of BTMs and the metabolic activity of osteoblasts quantified by radiomic analysis of thoroughbred racehorse scintigraphy:
Group 1 will comprise 50 thoroughbred racehorses referred for equine scintigraphy (bone scan) in the clinical investigation of lameness or poor performance to the TeleMedVET imaging centre, in Perth, Western Australia and 50 horses referred to the Hong Kong Jockey Club, Sha Tin, New Territories, Hong Kong; and
Group 2 will comprise 100 healthy, sound thoroughbred racehorses (50 from Perth and 50 from Hong Kong) matched for age and breed to Group 1.

Following ethics approval, consent will be obtained from trainers to collect a blood sample from each horse prior to commencing the study so that all study subjects will have a blood sample and bone scan performed.

### Methods

This is a prospective observational case control study that will concurrently measures serum bone and cartilage biomarkers (BTM) and radiomic markers of radionuclide uptake in bone and joints of racehorses of two groups of horses. Group 1 represent clinical cases referred for lameness investigations and Group 2 represent sound horses from the same population matched by age, breed and occupation which are used for research as controls (FIG. 1). Combined, approximately 150 scans/annum are undertaken at the sites for the investigation of lameness or poor performance. The minimum required to reach statistical power (calculation outlined below) is 36 and a target of 50 cases per site will further lower the probability of false negative error.

Both groups of recruited horses will undergo a full clinical veterinary examination prior to being admitted. For Group 1, this will include a movement asymmetry examination without nerve blocks and a quantitative assessment using the Equinosis Lameness Locator system (https://equinosis.com). Relevant past medical history and medications will be recorded. Horses that are unwell (fever, inappetence, colic, pneumonia, etc), had clinical signs of a synovial infection, had NSAID medication in the last 7 days or any intra-articular medication in the last 14 days will be excluded from the study. Each subject will be stabled immediately adjacent to the scanning centre, with clinical staff available 24 hours per day.

Blood sampling, determination of serum biomarkers, imaging, imaging analysis and statistical analysis will be performed as described in Example 1 above with variations or additional details set out below.

### Blood Samples

In Perth, blood samples will be refrigerated and transported to the Hormone Analysis Laboratory, School of Agriculture and Environment, University of Western Australia (UWA) within 6 hours of collection. In Hong Kong, identical sampling technique will be performed at Sha Tin and frozen serum samples will be batched by air courier to UWA to ensure procedural consistency and limit inter-laboratory variability.

### Equine Nuclear Planar and 3D-SPECT Scintigraphy

Group 1—Lame horses

In Perth, nuclear scintigraphy will be conducted by scientists and veterinarians licenced to perform equine studies in accordance with the Radiation Safety Act, Western Australia, at registered premises for the handling and storage of unsealed and sealed radioactive sources. In Hong Kong, nuclear scintigraphy will be conducted on a planar MiE Equine H.R. scinctron (Mie GmbH—Germany) by veterinarians licenced to perform equine studies in accordance with the Veterinary Surgeons Registration Ordinance and Hong Kong Radiation Ordinance at the Hong Kong Jockey Clubs Equine Hospital, which is a registered premises for the handling and storage of medical radioactive sources.

### Group 2—Control Horses

In Perth, local trainers operating in the Ascot racing locality near to the research centre have committed to providing healthy racehorses for data collection. These controls will be scheduled to minimise disruptions to training regimes and discharged 24 hours after the scan following veterinary approval. In Hong Kong, healthy racehorses at Sha Tin Racecource will be made available.

### Image Analysis

Radiologists will be asked to qualitatively categorise a scan to represent a region(s) of likely non-adaptative remodelling (positive case), equivocal (neutral) or unlikely (negative). Written reports of scan findings and interpretations for both groups will be generated for all racehorses in the study. Any significant features that are suspicious for impending fracture will be urgently relayed to trainers and their veterinarians.

### Statistical Power

The sample size calculation was based on OC data from Example 1. To calculate the effect size, required for the sample-size calculation, OC mean concentration and standard deviation data from control and lame horses were used. This was used to determine Cohen's d effect size and effect-size correlation (39). The effect-size correlation was calculated to be 0.54. Sample-size calculations were performed using G*Power 3.1 (version 3.1.9.6) software. Calculations were based on a two-tailed test, a type 1 error at the level of 5% (P=0.05). and a power of 80% *. An estimated total sample size of 36 was calculated.

1. A method for detecting pathological bone activity in an equine subject comprising:
(a) measuring osteocalcin level in a biological sample obtained from an equine subject; and
(b) comparing the osteocalcin concentration in the biological sample to a reference value,
wherein an elevated osteocalcin concentration in the biological sample relative to the reference value indicates the presence of pathological bone activity in the equine subject.

2. The method of claim 1, wherein an elevated osteocalcin concentration in the biological sample of at least 25% relative to the reference value indicates the presence of pathological bone activity in the equine subject.

3. The method of claim 1, wherein an elevated osteocalcin concentration in the biological sample of at least 50% relative to the reference value indicates the presence of pathological bone activity in the equine subject.

4. The method of claim 1, wherein an elevated osteocalcin concentration in the biological sample of at least 100% relative to the reference value indicates the presence of pathological bone activity in the equine subject.

5. The method of claim 1, wherein an osteocalcin concentration of greater than 0.85 ng/L in the biological sample indicates the presence of pathological bone activity in the equine subject.

6. The method of claim 1, wherein an osteocalcin concentration of greater than 1 ng/L in the biological sample indicates the presence of pathological bone activity in the equine subject.

7. The method of claim 1, wherein the pathological bone activity is present in at least one anatomical region of the equine subject selected from the group consisting of the pastern and hoof, fetlock, carpi, tarsi, tibiae, stifles, femora, hip joints, lower axial skeleton and upper axial skeleton.

8. The method of claim 1, further comprising obtaining at least one medical image to detect pathological bone activity in the equine subject.

9. The method of claim 8, wherein the medical image is a radiographic, scintigraphic or magnetic resonance image.

10. The method of claim 8, wherein the medical image is a SPECT/CT image.

11. The method of claim 1, wherein the equine subject is 2 years of age or more.

12. (canceled)

13. A method for predicting pathological bone fracture and/or lameness risk in an equine subject comprising:
(a) measuring osteocalcin level in a biological sample obtained from an equine subject;
(b) comparing the osteocalcin level in the biological sample to a reference value; and
wherein an elevated osteocalcin concentration in the biological sample relative to the reference value is indicative of a positive prediction of bone fracture and/or lameness risk if the subject is exposed to increased biomechanical force.

14. The method of claim 13, wherein an elevated osteocalcin concentration in the biological sample relative to the reference value is indicative of a 50% risk of bone fracture and/or lameness.

15. The method of claim 13, wherein an elevated osteocalcin concentration in the biological sample relative to the reference value is indicative of a 60% risk of bone fracture and/or lameness.

16. The method of claim 13, wherein an elevated osteocalcin concentration in the biological sample relative to the reference value is indicative of a 70% risk of bone fracture and/or lameness.

17. The method of claim 13, further comprising exposing the equine subject to a treatment regimen for preventing or delaying the onset of bone fracture and/or lameness in the equine subject.

18. A method for reducing risk of bone fracture and/or lameness in an equine subject comprising:
(a) measuring osteocalcin level in a biological sample obtained from an equine subject; and
(b) comparing the osteocalcin level in the biological sample to a reference value;
wherein, if the osteocalcin concentration in the biological sample is elevated relative to the reference value, exposure of the equine subject to biomechanical force is reduced or substantially avoided so as to reduce the risk of bone fracture and/or lameness.

19. The method of claim 18, further comprising exposing the equine subject to a further treatment regimen to reduce the risk of bone fracture and/or lameness.

20. The method of claim 18, which is repeated after one or more periods of time until the osteocalcin concentration in the biological sample obtained in step (a) is not elevated relative to the reference value, before exposing the equine subject to increased biomechanical force.

21. The method of claim 18, wherein the equine subject is a race horse.

22. The method of claim 21, wherein reducing or substantially avoiding exposure of the equine subject to biomechanical force involves scratching the race horse from a race.

23. A kit for detecting osteocalcin level in an equine biological sample comprising:
(a) a device for obtaining a biological sample from an equine subject;
(b) a test for measuring osteocalcin concentration in the biological sample relative to a reference value.

24. The kit of claim 23, further comprising a data processing and output device.
